(19) United States
(12) Patent Application Publication
(10) Pub. No.: US 2022/0148740 A1
(43) Pub. Date: May 12, 2022

(54) MEDICAL TREATMENT SUPPORT APPARATUS

(71) Applicant: CANON MEDICAL SYSTEMS CORPORATION, Tochigi (JP)

(72) Inventor: Yusuke KANO, Nasushiobara (JP)

(73) Assignee: CANON MEDICAL SYSTEMS CORPORATION, Tochigi (JP)

(21) Appl. No.: 17/451,887

(22) Filed: Oct. 22, 2021

(30) Foreign Application Priority Data
Nov. 9, 2020 (JP) 2020-186312

(51) Int. Cl. G16H 50/70; G16H 50/30; G16H 10/60

(52) U.S. Cl. CPC G16H 50/70; A61B 5/7275; G16H 10/60; G16H 50/30

(57) ABSTRACT

A medical treatment support apparatus according to one embodiment includes processing circuitry. The processing circuitry sets at least one reference object to be compared with a patient. The processing circuitry acquires a first index corresponding to an index expressing an influence of each of a plurality of candidate treatments for a disease on the patient, and a second index corresponding to an index expressing an influence of each of the candidate treatments on the reference object. The processing circuitry derives a relative index of the first index of each of the candidate treatments with respect to the second index. The processing circuitry presents the relative index of each of the candidate treatments.

### CROSS-REFERENCE TO RELATED APPLICATIONS

This application is based upon and claims the benefit of priority from Japanese Patent Application No. 2020-186312, filed on Nov. 9, 2020; the entire contents of which are incorporated herein by reference.

### FIELD

Embodiments described herein relate to a medical treatment support apparatus.

### BACKGROUND

In a case where a patient does not fully accept his disease or prognosis, for example, the patient and his doctor may disagree with each other. For example, in a case where the doctor thinks that medication or surgery is advantageous in improving the prognosis over no treatment about the patient's disease while the patient thinks that the treatment over many years does not make a large difference so causing less pain is more important, the doctor and the patient may disagree with each other.

### BRIEF DESCRIPTION OF THE DRAWINGS

FIG. 1 is a diagram illustrating one example of a structure of a medical treatment support system including a medical treatment support apparatus according to a first embodiment;

FIG. 2 is a diagram for describing a problem to be solved;

FIG. 3 is a diagram for describing a problem to be solved;

FIG. 4 is a flowchart illustrating a procedure of a process by the medical treatment support apparatus according to the first embodiment;

FIG. 5 is a diagram illustrating one example of a screen to be displayed on a terminal in the first embodiment;

FIG. 6 is a diagram illustrating one example of a screen to be displayed on the terminal in the first embodiment;

FIG. 7 is a diagram illustrating one example of a screen to be displayed on the terminal in the first embodiment;

FIG. 8 is a diagram illustrating one example of a screen to be displayed on the terminal in the first embodiment;

FIG. 9 is a diagram for describing a process by the medical treatment support apparatus according to a second embodiment;

FIG. 10A is a diagram illustrating one example of the screen to be displayed on the terminal in the second embodiment;

FIG. 10B is a diagram illustrating one example of the screen to be displayed on the terminal in the second embodiment;

FIG. 11A is a diagram illustrating one example of the screen to be displayed on the terminal in the second embodiment;

FIG. 11B is a diagram illustrating one example of the screen to be displayed on the terminal in the second embodiment;

FIG. 12A is a diagram illustrating one example of the screen to be displayed on the terminal in the second embodiment;

FIG. 12B is a diagram illustrating one example of the screen to be displayed on the terminal in the second embodiment;

FIG. 13A is a diagram illustrating one example of the screen to be displayed on the terminal in the second embodiment;

FIG. 13B is a diagram illustrating one example of the screen to be displayed on the terminal in the second embodiment;

FIG. 14A is a diagram illustrating one example of the screen to be displayed on the terminal in the second embodiment; and

FIG. 14B is a diagram illustrating one example of the screen to be displayed on the terminal in the second embodiment.

### DETAILED DESCRIPTION

A medical treatment support apparatus according to one embodiment includes processing circuitry. The processing circuitry sets at least one reference object to be compared with a patient. The processing circuitry acquires a first index corresponding to an index expressing the influence of each of a plurality of candidate treatments for a disease on the patient, and a second index corresponding to an index expressing the influence of each of the candidate treatments on the reference object. The processing circuitry derives a relative index of the first index of each of the candidate treatments with respect to the second index. The processing circuitry presents the relative index of each of the candidate treatments.

### First Embodiment

Embodiments of the medical treatment support apparatus are hereinafter described in detail with reference to the attached drawings. A medical treatment support system including the medical treatment support apparatus is hereinafter described as one example.

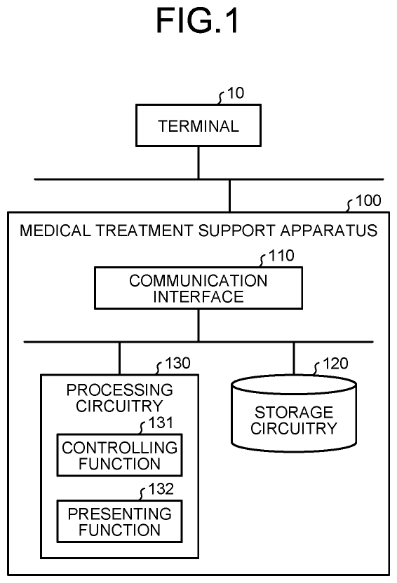

FIG. 1 is a diagram illustrating one example of a structure of a medical treatment support system 1 including a medical treatment support apparatus 100 according to an embodiment. The medical treatment support system illustrated in FIG. 1 includes a medical treatment support apparatus 100 and a terminal 10. The medical treatment support apparatus 100 performs communication with the terminal 10.

For example, the terminal 10 includes a personal computer (PC), a tablet type PC, a personal digital assistant (PDA), a mobile terminal, and the like. The terminal 10 is provided in a hospital and used by a patient's doctor.

The medical treatment support apparatus 100 includes a communication interface 110, storage circuitry 120, and processing circuitry 130.

The communication interface 110 is connected to the processing circuitry 130, and controls the communication and the transmission of various kinds of data between the medical treatment support apparatus 100 and the terminal 10.

The storage circuitry 120 is connected to the processing circuitry 130, and stores various kinds of data therein. For example, the storage circuitry 120 is achieved by a semiconductor memory element such as a RAM or a flash memory, a hard disk, an optical disk, or the like. Note that the storage circuitry 120 is one example of a means that achieves a storage unit. Moreover, the storage circuitry 120 is not necessarily incorporated in the medical treatment support apparatus 100 as long as the medical treatment support apparatus 100 can be accessed on a network.

The storage circuitry 120 stores a plurality of pieces of patient information from electronic medical records created for each of a plurality of patients, for example. Each piece of patient information includes basic information and medical treatment data of the patient. The basic information includes identification information that identifies the patient, name, birthday, sex, blood type, height, weight, and the like. The medical treatment data includes information about numerals (measurement values), the medical records, and the like and information expressing the recording date. Examples of the medical treatment data include prescription data, nurse's record data, and the like. The prescription data is the medical treatment data about the prescription. The nurse's record data is the medical treatment data about the nurse's record.

Note that the data and the information stored in the storage circuitry 120 and used in the embodiment are described below.

The processing circuitry 130 controls the components of the medical treatment support apparatus 100. For example, the processing circuitry 130 performs a controlling function 131 and a presenting function 132 as illustrated in FIG. 1. Here, for example, the processing functions performed by the controlling function 131 and the presenting function 132, which are the components of the processing circuitry 130, are recorded in the storage circuitry 120 as computer-executable computer programs. The processing circuitry 130 is a processor that reads out each computer program from the storage circuitry 120 and executes the computer program, thereby achieving the function corresponding to the computer program. In other words, the processing circuitry 130 that has read out the computer program has the corresponding function in the processing circuitry 130 illustrated in FIG. 1.

The medical treatment support apparatus 100 has a display application (computer program) implemented therein, and the display application can be read out by the terminal 10. For example, a user of the terminal 10 can cause a display of the terminal to display the display data transmitted from the medical treatment support apparatus 100 using the display application read out by the terminal. Note the controlling function 131 is one example of a setting unit, an acquisition unit, and a deriving unit. The presenting function 132 is one example of a presentation unit.

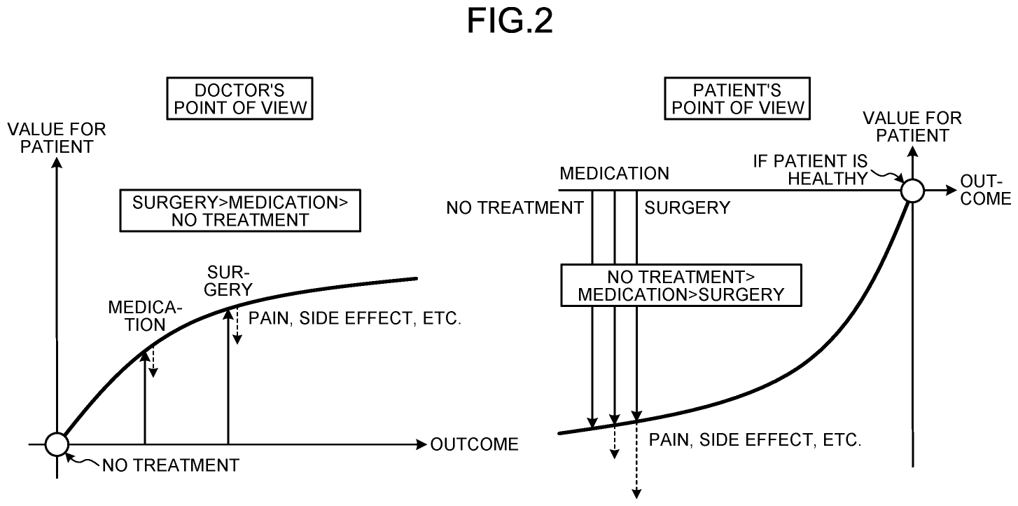

The term “processor” used in the above description refers to a circuit such as a central processing unit (CPU), a graphics processing unit (GPU), an application specific integrated circuit (ASIC), or a programmable logic device (for example, a simple programmable logic device (SPLD), a complex programmable logic device (CPLD), or a field programmable gate array (FPGA)). If the processor is a CPU, for example, the processor can achieve the function by reading out and executing the computer program saved in the storage circuitry 120. On the other hand, if the processor is an ASIC, for example, the computer program is not saved in the storage circuitry 120 but the computer program is incorporated directly in the circuitry of the processor. Note that each processor in the present embodiment is not limited to a structure formed as a single circuit for each processor, and a plurality of independent circuits may be combined and formed as one processor to achieve the function. Furthermore, a plurality of components illustrated in FIG. 2 may be integrated into one processor to achieve the function.

The overall structure of the medical treatment support system including the medical treatment support apparatus 100 according to the first embodiment is described above. With this structure, the medical treatment support apparatus 100 prevents the doctor and the patient from disagreeing with each other.

For example, in the case where the patient does not fully accept his disease or prognosis, the patient and the doctor may disagree with each other. The reason can be explained based on diminishing sensitivity and reference dependence that are the principles of the prospect theory, which is the representative theory of behavioral economics. The diminishing sensitivity refers to the characteristic that as the absolute value of the loss and gain increases, the sensitivity decreases, and the reference dependence refers to the characteristic that when a person judges the loss and gain, he establishes the value on the basis of the magnitude of the change from a certain point rather than from the absolute standard.

For example, in a case where the doctor thinks that medication or surgery is advantageous in improving the prognosis over no treatment about the patient's disease, performing the medication or surgery is worth the loss such as pain in the surgery or a side effect from the medication from the doctor's point of view. In this case, as illustrated in FIG. 2, “value for patient” increases in the order from “no treatment”, “medication”, and “surgery” from the doctor's point of view. On the other hand, in a case where the patient expects to live ten more years and long-term treatment only extends six months or a year so this is not a large difference, he may want to choose the option that is less painful. In this case, from the patient's point of view, the patient wants to choose no treatment over medication or surgery. In this case, as illustrated in FIG. 2, “value for patient” increases in the order from “surgery”, “medication”, and “no treatment” from the patient's point of view. In this case, the doctor and the patient are likely to disagree with each other.

In a case where the patient already feels pain for the disease or the treatment, the doctor and the patient may disagree with each other. The reason can be explained based on the loss aversion and the reference dependence that are the principles of the prospect theory of behavioral economics. The loss aversion refers to the characteristic that a person feels more impact from the pain due to loss than from the gain.

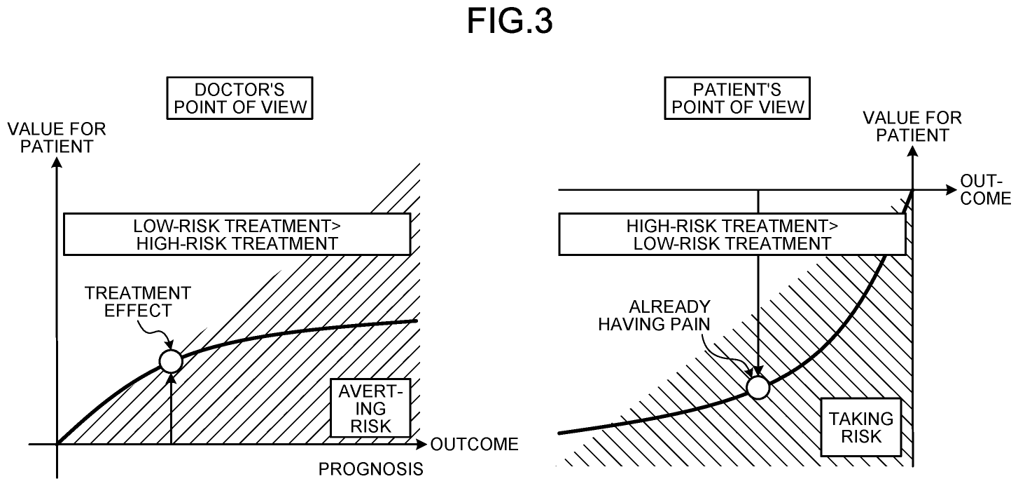

For example, in a case where the doctor thinks that the patient has tried the treatment so far and doing the high-risk treatment here may waste the patient's long effort, that is, the doctor wants to perform the treatment averting the risk, the treatment with the low risk is valuable for the patient from the doctor's point of view. In this case, as illustrated in FIG. 3, “low-risk treatment” is over “high-risk treatment” in “value for patient” from the doctor's point of view. On the other hand, for example, in a case where the patient thinks that he has already suffered thoroughly so he wants to be rewarded with the treatment effects for the pains that he has felt so far even if the treatment may fail, that is, in a case where the patient wants to obtain the treatment effect even if there is a risk, it is valuable to perform the high-risk treatment from the patient's point of view. In this case, “high-risk treatment” is over “low-risk treatment” in “value for patient” from the patient's point of view as illustrated in FIG. 3. In this case, the doctor and the patient are likely to disagree with each other.

Thus, the doctor and the patient put their priority on different things and think differently. Accordingly, it is necessary that the doctor and the patient have the same sense of values as much as possible.

In view of the above, the medical treatment support apparatus 100 according to the first embodiment performs the following process. First, in the medical treatment support apparatus 100 according to the first embodiment, the controlling function 131 sets at least one reference object to be compared with the patient. The controlling function 131 acquires the first index corresponding to the index expressing the influence of each of the candidate treatments for the disease on the patient, and the second index corresponding to the index expressing the influence of each of the candidate treatments on the reference object. The controlling function 131 calculates the relative index of the first index of each of the candidate treatments with respect to the second index. The presenting function 132 presents the relative index of each of the candidate treatments.

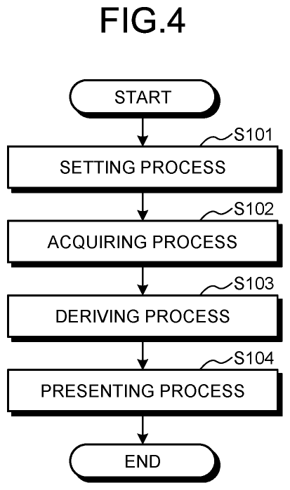

The respective functions of the controlling function 131 and the presenting function 132 are hereinafter described with reference to FIG. 4 to FIG. 8. FIG. 4 is a flowchart illustrating a procedure of the process by the medical treatment support apparatus 100 according to the first embodiment.

Step S101 in FIG. 4 is a step where the processing circuitry 130 calls a computer program corresponding to the controlling function 131 from the storage circuitry 120 and executes the computer program. At step S101, the controlling function 131 performs a setting process. Specifically, the controlling function 131 sets at least one reference object to be compared with the patient P.

Here, in the present embodiment, for example, the storage circuitry 120 stores therein treatment influence information in addition to the aforementioned pieces of patient information. The treatment influence information is the information about the influence of each of the treatment plans for the disease in a particular person or a particular group. The particular person represents a person similar to the patient P, for example. The particular group expresses, for example, a patient group with the same attribute as the patient P or a group including persons not having the corresponding disease. For example, in a case where the disease of the patient P is aortic stricture, the treatment plan is surgery, catheterization, medication, no treatment, or the like. Moreover, the treatment influence information includes the information about the age group, sex, and the like of the particular person or the particular group.

First, in the setting process, the controlling function 131 sets a type of the index expressing the influence of each of the treatment plans for the disease on the patient P. For example, when the patient P's doctor designates the type of the index as the designation of the outcome using the terminal 10, the controlling function 131 sets the designated type of the index.

Here, since the patient P and the patient P's doctor need to have the same sense of values as much as possible, the outcome may be designated in accordance with the patient P's doctor, who is the user, the outcome may be designated in accordance with the patient P's request, or the outcome may be designated in accordance with the combination of the patient P and the patient P's doctor.

Examples of the types of the index include a survival rate, a death rate, survival period, quality-adjusted life year (QALY), an adverse event rate, a hospital readmission rate, and quality of life (QOL). The index may be the index about the loss. Examples of the index about the loss include a side effect, medical expense, and a treatment period. Here, for example, in the case of “three-year survival rate”, the survival rate represents the rate of patients who have survived three years among the group after three years from the surgery. In the case of “five-year death rate”, the death rate represents the rate of patients who died among the group after five years from the surgery. As for the survival rate, the reference date may be not just “surgery date” but also “diagnosis date”, “treatment start date”, “today (information presented date)”, or the like.

Next, in the setting process, the controlling function 131 sets at least one reference object to be compared with the patient P. For example, the patient P's doctor designates the reference object using the terminal 10, so that the controlling function 131 sets the designated reference object. Here, the reference object means the aforementioned particular person or the aforementioned particular group, and includes the information about the age group, sex, and the like of the particular person or the particular group. Note that the particular person includes a person selected arbitrarily. The particular group includes a group selected arbitrarily and a group of all people from which information can be acquired.

Step S102 in FIG. 4 is a step where the processing circuitry 130 calls a computer program corresponding to the controlling function 131 from the storage circuitry 120 and executes the computer program. At step S101, the controlling function 131 performs an acquiring process. Specifically, the controlling function 131 acquires the index expressing the influence of each of the candidate treatments for the disease on the patient P, and the index expressing the influence of each of the candidate treatments on the reference object. Here, the index expressing the influence of each of the candidate treatments for the disease on the patient P is one example of “the first index”, and the index expressing the influence of each of the candidate treatments on the reference object is one example of “the second index”.

First, in the acquiring process, the controlling function 131 acquires, from the treatment influence information, the index expressing the influence of each of the candidate treatments for the disease on the patient P on the basis of the type of the index designated by the patient P's doctor using the terminal 10. Specifically, the controlling function 131 predicts the index of each of the candidate treatments about the patient P from the treatment influence information of the patient group with the same attribute as the patient P. For example, the controlling function 131 predicts the index of each of the candidate treatments about the patient P by a statistic process, or by using a learned model obtained from machine learning.

Next, in the acquiring process, the controlling function 131 calculates the index expressing the influence of each of the candidate treatments for the disease on the reference object on the basis of the reference object designated by the patient P's doctor using the terminal 10.

Step S103 in FIG. 4 is a step where the processing circuitry 130 calls a computer program corresponding to the controlling function 131 from the storage circuitry 120 and executes the computer program. At step S103, the controlling function 131 performs a deriving process. Specifically, the controlling function 131 derives the relative index of the index of each of the candidate treatments relative to the index of the reference object.

For example, the controlling function 131 derives, as the relative index, any of the ratio, the dispersion, and the difference of the value of the index of each of the treatment plans with respect to the index of the reference object.

Note that the controlling function 131 may derive the relative index of the index of each of the candidate treatments after excluding candidate treatments that are unsuitable for the patient P. For example, if the drug to be used in the medication for the disease is incompatible with the drug taken by the patient P for his basal disease, the controlling function 131 excludes the medication. In this case, the controlling function 131 calculates the relative index of the index of each of the candidate treatments after excluding the candidate treatment corresponding to the treatment plan that is unsuitable for the patient P among the candidate treatments with respect to the index of the reference object.

Step S104 in FIG. 4 is a step where the processing circuitry 130 calls a computer program corresponding to the presenting function 132 from the storage circuitry 120 and executes the computer program. At step S104, the presenting function 132 performs a presenting process. Specifically, the presenting function 132 presents the relative index of each of the candidate treatments.

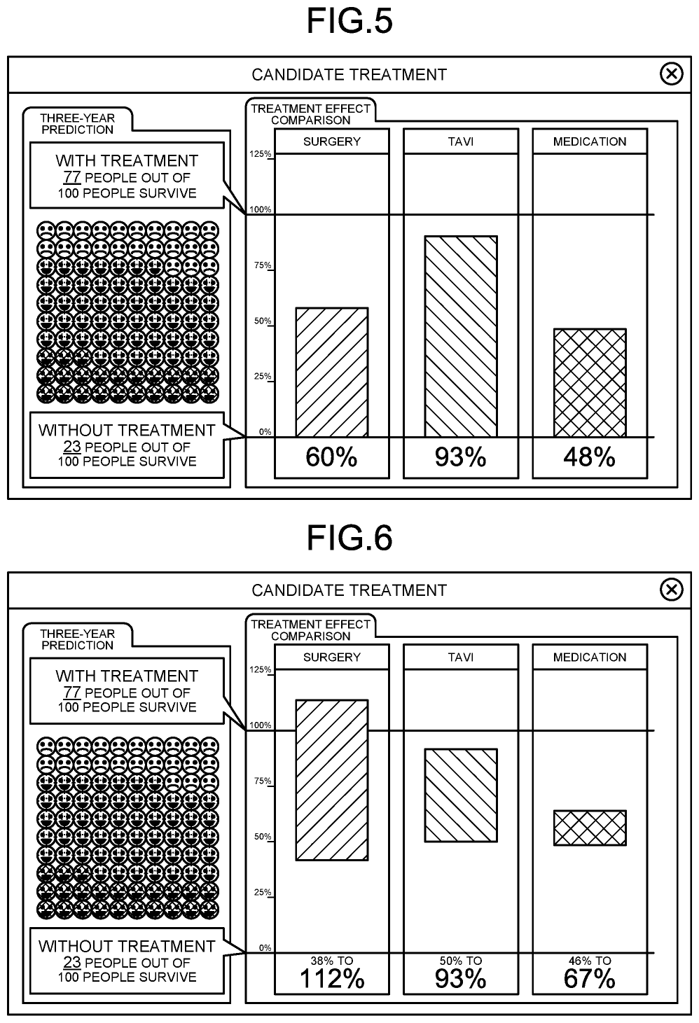

FIG. 5 to FIG. 8 each illustrate one example of the screen to be displayed on the terminal 10 of the patient P's doctor. For example, it is assumed that, in the setting process, upon the designation from the terminal 10, the controlling function 131 sets “three-year prediction (three-year survival rate)” as the type of the index, and sets, as the reference objects, two groups including a patient group “with treatment” having been subjected to various standard treatments for the disease including “surgery”, “catheterization” (hereinafter referred to as “TAVI”), “medication”, and the like and a patient group “without treatment” not having been subjected to any treatment for the disease. The patient group “without treatment” includes, for example, patients with follow-up only. In this case, in the acquiring process, the controlling function 131 predicts the index “three-year prediction” expressing the influence of each of the candidate treatments for the disease, “surgery”, “TAVI”, and “medication” on the patient P. Moreover, the controlling function 131 calculates the index of the reference object.

Here, for example, the controlling function 131 derives, as the relative index, the ratio of each value of the indexes of the candidate treatments “surgery”, “TAVI”, and “medication” relative to the index of the reference object. The designation to derive the index values by the ratio is performed by the patient P's doctor through the operation of the terminal 10. Here, the presenting function 132 causes the display of the terminal 10 to display the screen illustrated in FIG. 5 as the relative indexes of the candidate treatments “surgery”, “TAVI”, and “medication”.

In the display on the screen illustrated in FIG. 5, the index “three-year prediction” indicates that 77 people out of 100 people survive among the patient group “with treatment” having been subject to the treatment, and 23 people out of 100 people survive among the patient group “without treatment” not having been subject to the treatment. The screen illustrated in FIG. 5 displays the relative ratios “60%”, “93%”, and “48%” of the representative values (average value, median value, etc.) of the indexes predicted for “surgery”, “TAVI”, and “medication” respectively as the relative indexes of the patient P in a case where the three-year survival rate of “without treatment” is defined as 0% and the three-year survival rate of “with treatment” is defined as 100%. The screen illustrated in FIG. 5 indicates that the survival possibility is high when the disease is treated in consideration of three years later, and in the case of performing the treatment, the catheterization is highly likely to be effective. In this case, for example, when the patient P who wants to choose the no treatment over the treatment such as surgery sees the screen in FIG. 5, he understands the effect of the catheterization. Thus, it is possible that the patient agrees with the doctor who thinks that performing the medication or surgery is worth the loss such as pain from the surgery or the side effect in the medication. That is to say, by sharing the sensitivity about the gain and loss between the doctor and the patient P, the doctor and the patient P may agree with each other.

For example, the controlling function 131 derives, as the relative indexes, the dispersion of the values of the respective indexes of the candidate treatments “surgery”, “TAVI”, and “medication” with respect to the index of the reference object. The designation to derive the index values by the dispersion is performed by the patient P's doctor through the operation of the terminal 10. Here, the presenting function 132 causes the display of the terminal 10 to display the screen illustrated in FIG. 6 as the relative indexes of the candidate treatments “ surgery”, “TAVI”, and “medication”.

In this case, the screen illustrated in FIG. 6 displays, as the relative indexes, the dispersion of the values of the respective indexes of the candidate treatments “surgery”, “TAVI”, and “medication” with respect to the index of the reference object. The screen illustrated in FIG. 6 displays that, in the case where the three-year survival rate of “without treatment” is defined as 0% and the three-year survival rate of “with treatment” is defined as 100%, the three-year survival rate that is expected if the patient P has “surgery” is in the range of “38 to 112%” relative to the three-year survival rate of “with treatment” that is “100%”. Similarly, the screen illustrated in FIG. 6 displays that the three-year survival rate that is expected if the patient P has “TAVI” is in the range of “50 to 93%” relative to the three-year survival rate of “with treatment” that is “100%”. Similarly, the screen illustrated in FIG. 6 displays that the three-year survival rate that is expected if the patient P has “medication” is in the range of “46 to 67%” relative to the three-year survival rate of “with treatment” that is “100%”. In this case, the effect of “TAVI” and “medication” is confirmed only in the region below 100%. On the other hand, as for “surgery”, although the effect varies widely, the effect can be confirmed in the range from the region over 100% to the region below 100%. In this case, for example, when the patient P who used to think the low-risk treatment is valuable sees the screen in FIG. 6, he may want to have the surgery, and the doctor and the patient P may agree with each other.

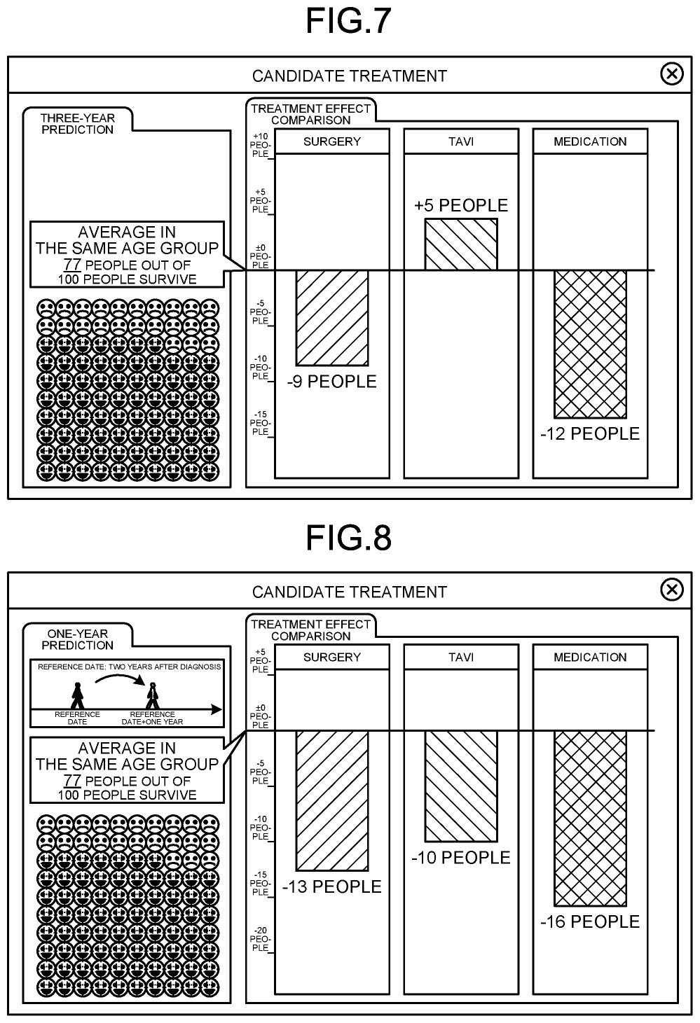

The reference object may be just one, and the relativity may be not just the ratio but the difference. Specifically, it is assumed that in the setting process, upon the designation from the terminal 10, the controlling function 131 sets “three-year prediction” as the type of the index and sets just the patient group “with treatment” having been subjected to some standard treatment for the disease as the reference object. For example, the controlling function 131 derives, as the relative index, the difference of the value of the index of each of the candidate treatments “surgery”, “TAVI”, and “medication” with respect to the index of the reference object. The designation to derive the index values by the difference is performed by the patient P's doctor through the operation of the terminal 10. Here, the presenting function 132 causes the display of the terminal 10 to display the screen illustrated in FIG. 7 as the relative indexes of the candidate treatments “surgery”, “TAVI”, and “medication”.

In the screen illustrated in FIG. 7, the differences “−9 people”, “+5 people”, and “−12 people” of the respective index values of the candidate treatments “surgery”, “TAVI”, and “medication” with respect to the index of the reference object are displayed as the relative indexes. In the screen illustrated in FIG. 7, in a case where the average in the group “with treatment” is “77 people out of 100 people survive” after three years, since the prediction index in the case of the patients P “with the surgery” indicates “68 people out of 100 people survive”, “−9 people” is displayed. Similarly, since the prediction index in the case of the patients P “with TAVI” indicates “82 people out of 100 people survive”, the screen illustrated in FIG. 7 displays “+5 people”. Similarly, since the prediction index in the case of the patients P with “medication” indicates “65 people out of 100 people survive”, the screen illustrated in FIG. 7 displays “−12 people”. In this case, maximizing the sensitivity about the gain and loss is effective, for example, for the patient P who thinks the treatment with the higher treatment effect than the average is valuable, and the doctor and the patient P may agree with each other.

The controlling function 131 can set the temporal parameter about the index. Specifically, upon the reception of the change of the type of the index, the controlling function 131 acquires the index of the type after the change. For example, when “reference date” is changed from “diagnosis date” to “two years after diagnosis” by the setting from the terminal 10 while the difference of “three-year prediction” is displayed in FIG. 7, the index is changed from “three-year prediction” to “one-year prediction” of “two years after diagnosis”. In other words, the index is changed, for example, from the three-year survival rate to the rate that people who have survived two years after the diagnosis are still alive one year later (one-year survival rate of two-year survivor).

In the display on the screen illustrated in FIG. 8, the index “one-year prediction (of two-year survivor)” indicates that 77 people out of 100 people survive among the patient group “in the same age group” and “with treatment” having been subjected to the treatment. In the screen illustrated in FIG. 8, in the case where the average among the group “in the same age group” and “with treatment” indicates “77 people out of 100 people survive” three years later, since the prediction index in the case of the patients P with “surgery” indicates “64 out of 100 people survive”, “−13 people” is displayed. Similarly, the prediction index in the case of the patients P with “TAVI” indicates “67 people out of 100 people survive” on the screen in FIG. 8; therefore, “−10 people” is displayed. Similarly, the prediction index in the case of the patients P with “medication” indicates “61 people out of 100 people survive” on the screen in FIG. 8; therefore, “−16 people” is displayed. In this case, in the case where the influence of the gain and the loss in the near future is too large, suppressing this influence may make it possible for the doctor and the patient P to agree with each other.

As described above, in the medical treatment support apparatus 100 according to the first embodiment, the controlling function 131 sets at least one reference object to be compared with the patient P, and acquires the first index corresponding to the index expressing the influence of each of the candidate treatments for the disease on the patient P, and the second index corresponding to the index expressing the influence of each of the candidate treatments for the disease on the reference object. The controlling function 131 derives the relative index of the first index of each of the candidate treatments with respect to the second index and the presenting function 132 presents the relative index of each of the candidate treatments. Therefore, the doctor and the patient P can have the same sense of value as much as possible. Accordingly, the medical treatment support apparatus 100 according to the first embodiment can prevent the doctor and the patient P from disagreeing with each other.

For example, the explanation can be made based on the diminishing sensitivity and the reference dependence from the prospect theory, which is the representative theory of the behavioral economics. When the presenting function 132 presents the relative index of each of the candidate treatments to the patient P and the patient P's doctor, the doctor who thinks that performing the medication or surgery is worth the loss such as pain from the surgery or the side effect in the medication, and the patient P who wants to choose no treatment over the treatment such as surgery may agree with each other.

Moreover, the explanation can be made based on the loss aversion and the reference dependence from the prospect theory of the behavioral economics. When the presenting function 132 presents the relative index of each of the candidate treatments to the patient P and the patient P's doctor, the doctor who thinks performing the low-risk treatment is valuable for the patient, and the patient who thinks performing the high-risk treatment is valuable may agree with each other.

Thus, by making the doctor and the patient P share the sensitivity about the gain and loss, the doctor and the patient P may agree with each other. Therefore, in the medical treatment support apparatus 100 according to the first embodiment, based on the relative positional relation at the axis of the outcome between the index value of the reference object (reference point) and the index value predicted for each of the candidate treatments about the patient, the reference object and the type of the index that make the patient want to agree on the doctor's suggestion are set and the result is presented. Thus, in the first embodiment, it is possible to prevent the doctor and the patient P from disagreeing with each other.

### Second Embodiment

In the medical treatment support apparatus 100 according to a second embodiment, reference candidate objects to become the reference objects are extracted from the candidate treatments chosen by the doctor corresponding to the user (patient P's doctor) and the reference object chosen by the patient P's doctor from the reference candidate objects is set.

First, the controlling function 131 acquires the representative values (average value, median value, etc.) of the indexes expressing the influence of “surgery”, “TAVI”, and “medication” for the disease on the patient, or the variation, for the respective indexes from the treatment influence information. Examples of the indexes include “three-year prediction (three-year survival rate)”, “five-year prediction (five-year survival rate)”, and “one-year prediction of two-year survivor (one-year survival rate)”.

Next, the controlling function 131 acquires the candidates of the reference object regarding each index. For example, a plurality of combinations chosen from the reference objects are the reference candidate objects. The reference objects include the patient group “without treatment” not having been subjected to the treatment such as “surgery”, “TAVI”, or “medication” for the disease, the patient group “with treatment” having been subjected to the treatment such as “surgery”, “TAVI”, or “medication” for the disease, and the like. When “the same age group” is the reference point, the reference objects include the patient group “in the same age group and without treatment” not having been subjected to the treatment such as “surgery”, “TAVI”, or “medication” for the disease, the patient group “in the same age group and with treatment” having been subjected to the treatment such as “surgery”, “TAVI”, or “medication” for the disease, and the like.

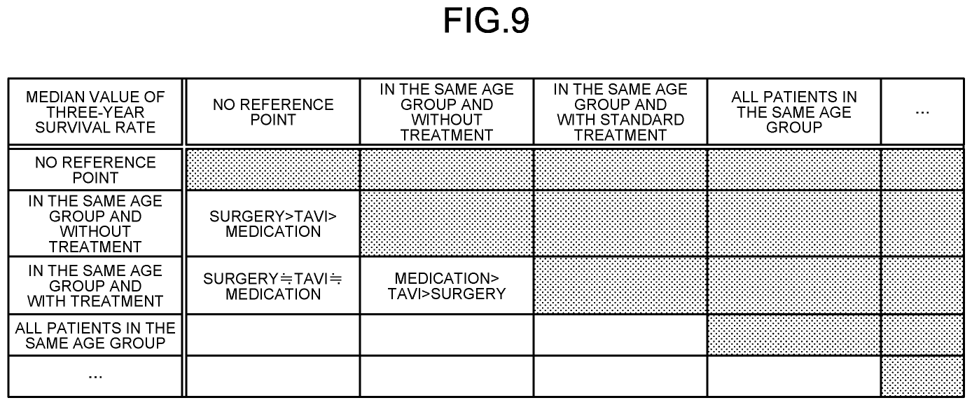

Next, the controlling function 131 derives the relative index for each of the reference candidate objects in each index and creates a table illustrated in FIG. 9. The table illustrated in FIG. 9 is created for each type of the index. For example, in the case where the index is “three-year prediction (three-year survival rate)” and the representative value of the index (average value, median value, etc.) is designated, the relative index as illustrated in FIG. 9 is derived. For example, the patient group “in the same age group and without treatment” has a difference in relative index compared to the patient group “without treatment” in the case where “the same age group” is not used as the reference point, and the index is higher in the order of “surgery”, “TAVI”, and “medication”. In addition, for example, the patient group “in the same age group and with treatment” has no difference in relative index compared to the patient group “with treatment” in the case where “the same age group” is not used as the reference point. Furthermore, the patient group “in the same age group and with treatment” has a difference in relative index compared to the patient group “without treatment” in the case where “the same age group” is not used as the reference point, and the index is higher in the order of “medication”, “TAVI”, and “surgery”.

Next, the presenting function 132 causes the terminal 10 to display the table in FIG. 9, thereby presenting the table to the patient P's doctor. The patient P's doctor chooses the reference candidate object that suits the purpose of the patient P's doctor with reference to the relative indexes of the reference candidate objects in the table displayed on the terminal 10. Here, the controlling function 131 sets the reference candidate object chosen by the patient P's doctor as the reference object.

In the case where the reference object is set, the process similar to that in the first embodiment is performed and the presenting function 132 causes the terminal 10 to display the screen that the patient P's doctor wants to show the patient P. One display example of the screen is described below.

First, as the display example of the screen, the display example based on the diminishing sensitivity and the reference dependence from the prospect theory of the behavioral economics is described.

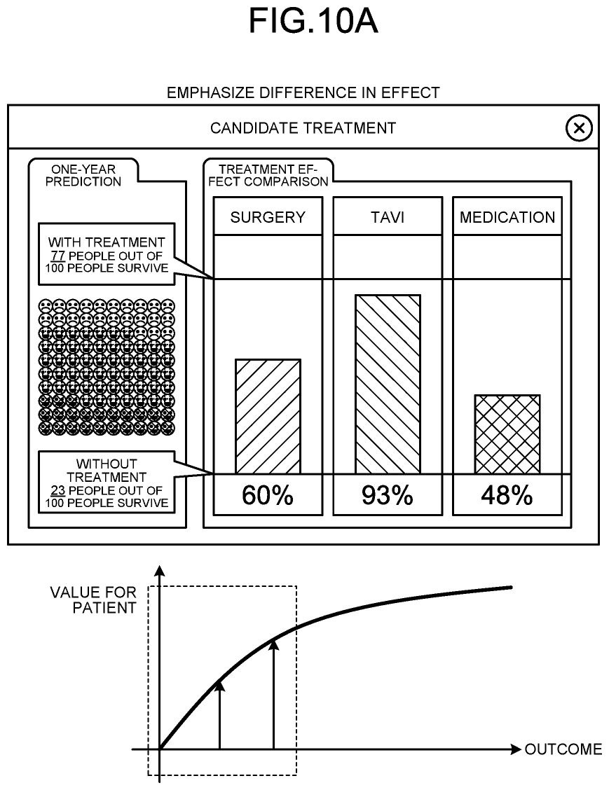

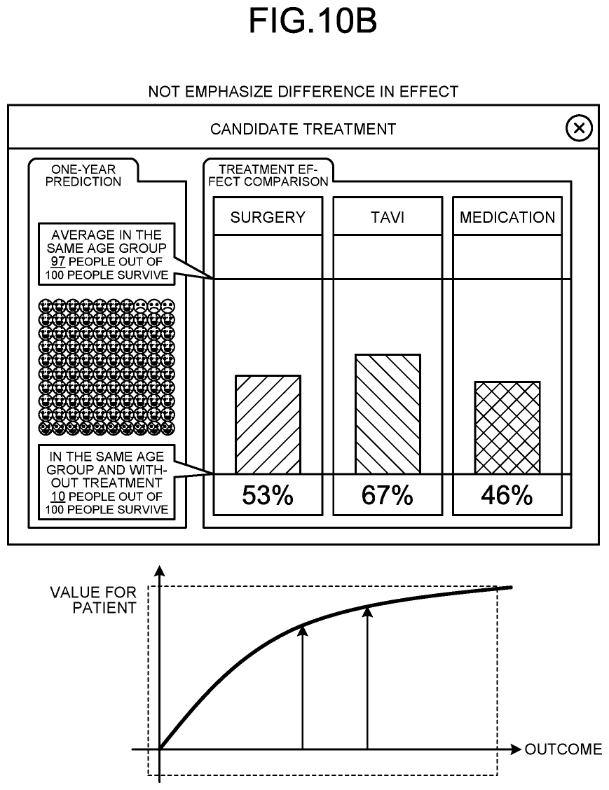

For example, in the case where the patient P's doctor wants to show the patient P the screen with emphasis on the difference in effect, the patient P's doctor designates “one-year prediction” as the type of the index, and designates two groups of the patient group “with treatment” having been subjected to the treatment such as “surgery”, “TAVI”, or “medication” for the disease, and the patient group “without treatment” not having been subjected to the treatment for the disease as the reference objects using the terminal 10. In this case, the controlling function 131 sets the reference candidate objects that suit the purpose of the patient P's doctor as the reference objects, and derives the values of the indexes of the reference candidate objects as the relative indexes. As a result, the presenting function 132 causes the display of the terminal 10 to display the screen illustrated in FIG. 10A as the relative indexes of the candidate treatments “surgery”, “TAVI”, and “medication”.

In the display on the screen illustrated in FIG. 10A, the index “one-year prediction” indicates that 77 people out of 100 people survive among the patient group “with treatment” having been subjected to the treatment and 23 people out of 100 people survive among the patient group “without treatment” not having been subjected to the treatment. The screen in FIG. 10A displays, as the relative indexes for the patient P in the case where the one-year survival rate of “without treatment” is defined as 0% and the one-year survival rate of “with treatment” is defined as 100%, the relative ratios “60%”, “93%”, and “48%” of the representative values (average value, median value, etc.) of the indexes predicted for “surgery”, “TAVI”, and “medication”. In this case, the effectiveness of “TAVI” is emphasized and for example, when the patient P who thinks the difference in effect is important sees the screen illustrated in FIG. 10A, the doctor and the patient P may agree with each other.

On the other hand, for example, in the case where the patient P's doctor wants to show the patient P the screen without emphasis on the difference in effect, the patient P's doctor designates “one-year prediction” as the type of the index and designates the patient group “in the same age group” who is in the same age group as the patient P among the patient group “with treatment” having been subjected to the treatment such as “surgery”, “TAVI”, or “medication” for the disease, as the reference object using the terminal 10. In this case, the controlling function 131 sets the reference candidate objects that suit the purpose of the patient P's doctor as the reference objects, and derives the value of the index of each reference candidate object as the relative index. As a result, the presenting function 132 causes the display of the terminal 10 to display the screen illustrated in FIG. 10B as the relative indexes of the candidate treatments “surgery”, “TAVI”, and “medication”.

In the display on the screen illustrated in FIG. 10B, the index “one-year prediction” indicates that 97 people out of 100 people survive among the patient group “average in the same age group” who is in the same age group and has been subjected to the treatment, and 10 people out of 100 people survive among the patient group “in the same age group and without treatment” who is in the same age group and has not been subjected to the treatment. The screen illustrated in FIG. 10B displays, as the relative indexes for the patient P in the case where the one-year survival rate of “in the same group and without treatment” is defined as 0% and the one-year survival rate of “average in the same group” is defined as 100%, the relative ratios “53%”, “67%”, and “46%” of the representative values (average value, median value, etc.) of the indexes predicted for “surgery”, “TAVI”, and “medication”. In this case, the difference in effect is not emphasized for “surgery”, “TAVI”, and “medication”.

Next, as the display example of the screen, a first display example based on the loss aversion and the reference dependence from the prospect theory of the behavioral economics is described.

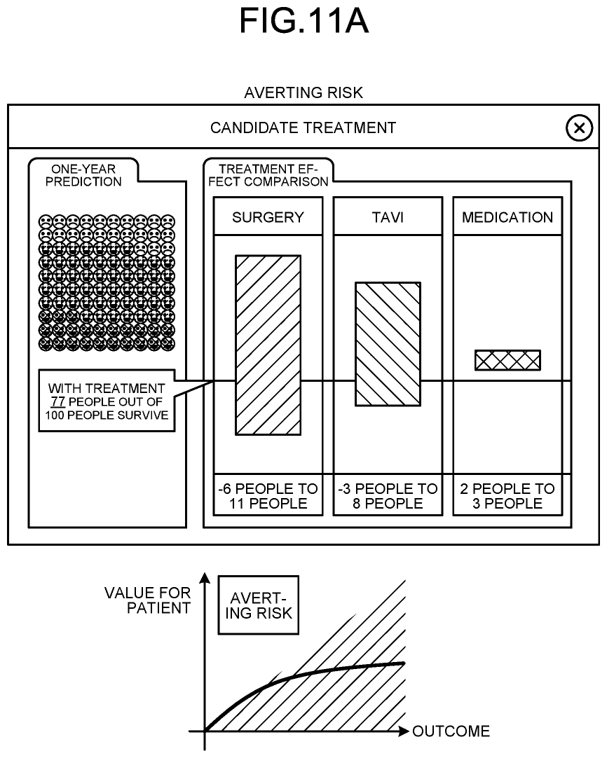

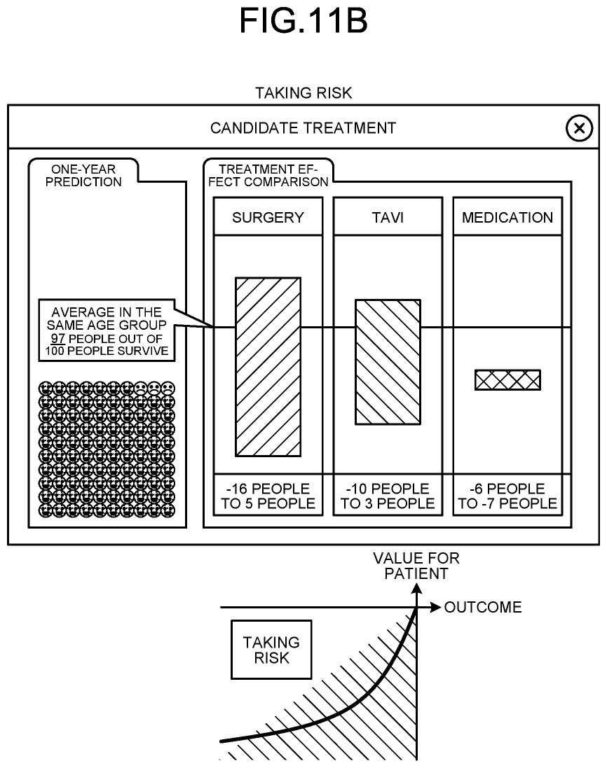

For example, in the case where the patient P's doctor wants to show the patient P the screen to recommend the low-risk treatment as the screen for averting the risk, the patient P's doctor designates “one-year prediction” as the type of the index, and designates two groups of the patient group “with treatment” having been subjected to the treatment such as “surgery”, “TAVI”, or “medication” for the disease, and the patient group “without treatment” not having been subjected to the treatment for the disease, as the reference objects using the terminal 10. As a result, the presenting function 132 causes the display of the terminal 10 to display the screen illustrated in FIG. 11A as the relative indexes of the candidate treatments “surgery”, “TAVI”, and “medication”.

In the display on the screen illustrated in FIG. 11A, the index “one-year prediction” indicates that 77 people out of 100 people survive among the patient group “with treatment” having been subjected to the treatment. In the display on the screen illustrated in FIG. 11A, the one-year survival rate predicted if the patient P has “surgery” is in the range of “−6 people to 11 people” with respect to the 77 survivors “with treatment”. Similarly, in the display on the screen illustrated in FIG. 11A, the one-year survival rate predicted if the patient P has “TAVI” is in the range of “−3 people to 8 people” with respect to the 77 survivors “with treatment”. Similarly, in the display on the screen illustrated in FIG. 11A, the one-year survival rate predicted if the patient P has “medication” is in the range of “2 people to 3 people” with respect to the 77 survivors “with treatment”. In this case, the effect of “medication” can be confirmed in the region over 100%. On the other hand, the effects of “surgery” and “TAVI”, although varying largely, can be confirmed in the range from the region much over 100% to the region below 100%. In this case, when the patient P who thinks the low-risk treatment is valuable sees the screen illustrated in FIG. 11A, the doctor and the patient P may agree with each other.

On the other hand, for example, in the case where the patient P's doctor wants to show the patient P the screen to recommend the high-risk treatment as the screen for taking the risk, the patient P's doctor designates “one-year prediction” as the type of the index, and designates the patient group in the same age group as the patient P among the patient group “with treatment” having been subjected to the treatment such as “surgery”, “TAVI”, or “medication” for the disease, as the reference object using the terminal 10. As a result, the presenting function 132 causes the display of the terminal 10 to display the screen illustrated in FIG. 11B as the relative indexes of the candidate treatments “surgery”, “TAVI”, and “medication”.

In the display on the screen illustrated in FIG. 11B, the index “one-year prediction” indicates that 97 people out of 100 people survive among the patient group “average in the same age group” who is in the same age group and has been subjected to the treatment. Moreover, the screen illustrated in FIG. 11B displays, as the relative indexes, the values of the indexes of the candidate treatments “surgery”, “TAVI”, and “medication” with respect to the index of the reference object. In addition, in the display on the screen illustrated in FIG. 11B, the one-year survival rate predicted if the patient P has “surgery” is in the range of “−16 people to 5 people” with respect to the 97 survivors “average in the same age group”. Similarly, in the display on the screen illustrated in FIG. 11B, the one-year survival rate predicted if the patient P has “TAVI” is in the range of “−10 people to 3 people” with respect to the 97 survivors “average in the same age group”. Similarly, in the display on the screen illustrated in FIG. 11B, the one-year survival rate predicted if the patient P has “medication” is in the range of “−6 people to −7 people” with respect to the 97 survivors “average in the same age group”. In this case, the effect of “medication” is confirmed only in the region below 100%. On the other hand, the effects of “surgery” and “TAVI”, although varying largely, can be confirmed in the range from the region much over 100% to the region much below 100%. In this case, when the patient P who thinks the high-risk treatment is valuable sees the screen illustrated in FIG. 11B, the doctor and the patient P may agree with each other.

Next, as the display example of the screen, a second display example based on the loss aversion and the reference dependence from the prospect theory of the behavioral economics is described.

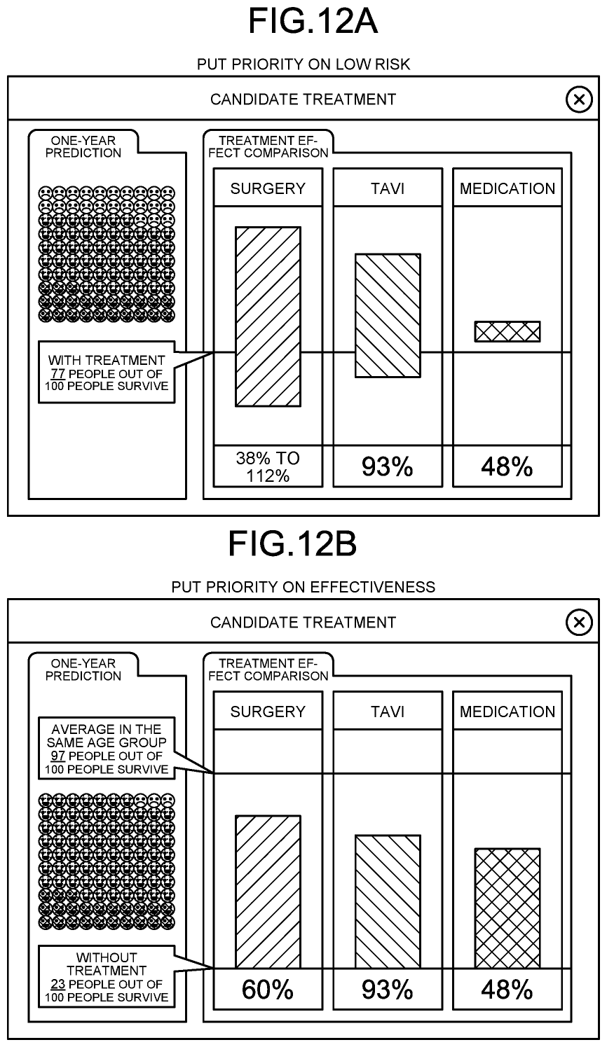

For example, in the case where the patient P's doctor wants to show the patient P the screen with the priority on the low risk on the basis of the loss aversion and the reference dependence from the prospect theory of the behavioral economics, the patient P's doctor designates “one-year prediction” as the type of the index, and designates the patient group “with treatment” having been subjected to the treatment such as “surgery”, “TAVI”, or “medication” for the disease, as the reference object using the terminal 10. As a result, the presenting function 132 causes the display of the terminal 10 to display the screen for averting the risk illustrated in FIG. 12A as the relative indexes of the candidate treatments “surgery”, “TAVI”, and “medication” in a manner similar to the case of FIG. 11A. In this case, for example, when the patient P who thinks the low-risk treatment is valuable sees the screen illustrated in FIG. 12A, the doctor and the patient P may agree with each other.

On the other hand, for example, the patient P's doctor may want to show the patient P the screen with the priority on the effectiveness. In this case, for example, the patient P's doctor designates “one-year prediction” as the type of the index, and designates two groups of the patient group in the same age group as the patient P among the patient group “with treatment” having been subjected to the treatment such as “surgery”, “TAVI”, or “medication” for the disease, and the patient group “without treatment” not having been subjected to the treatment for the disease, as the reference objects using the terminal 10. As a method of deriving the index, the ratio is designated from the terminal 10 by the operation of the patient P's doctor. As a result, the presenting function 132 causes the display of the terminal 10 to display the screen with emphasis on the difference in effect as illustrated in FIG. 12B as the relative indexes of the candidate treatments “surgery”, “TAVI”, and “medication” in a manner similar to the case of FIG. 10A. In this case, when the patient P who thinks the difference in effect is important sees the screen illustrated in FIG. 12B, the doctor and the patient P may agree with each other.

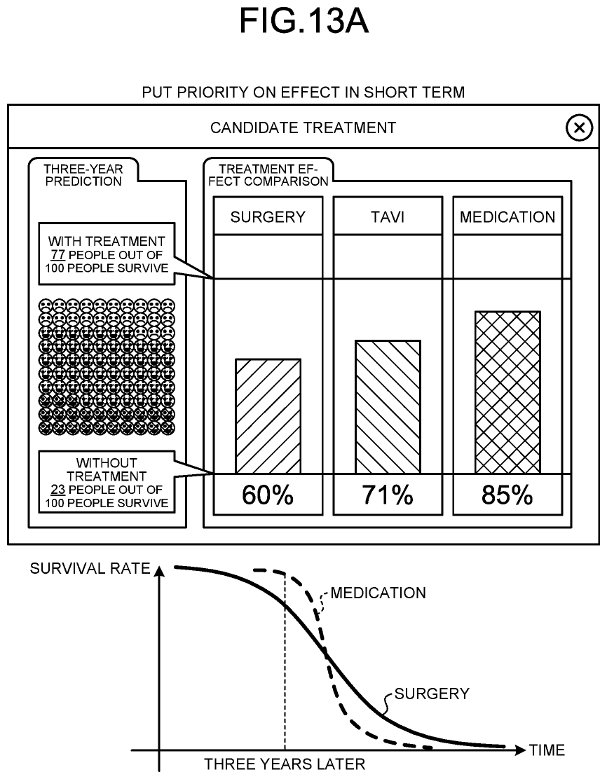

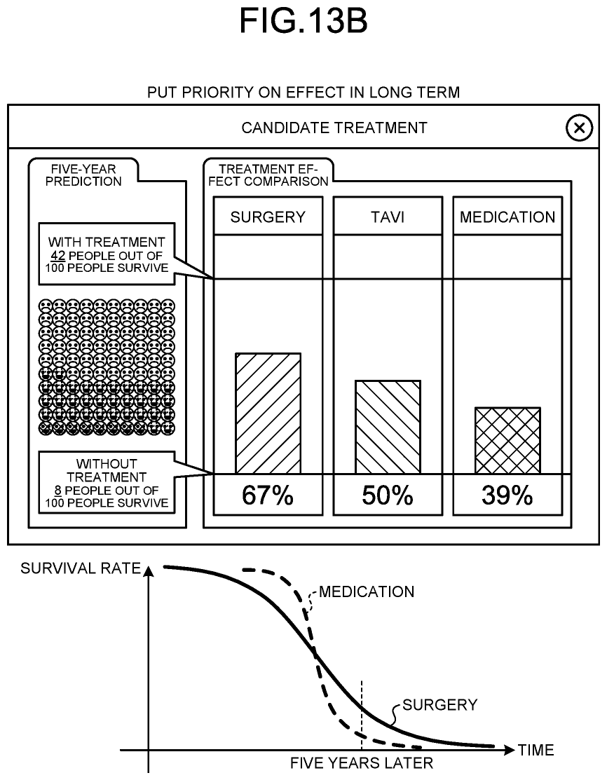

For example, in the case where the patient P's doctor wants to show the patient P the screen with the priority on the effect in the short term by changing the outcome, the patient P's doctor designates “three-year prediction” as the type of the index, and designates two groups of the patient group “with treatment” having been subjected to the treatment such as “surgery”, “TAVI”, or “medication” for the disease and the patient group “without treatment” not having been subjected to the treatment for the disease, as the reference objects using the terminal 10. As a method of deriving the index, the ratio is designated from the terminal 10 by the operation of the patient P's doctor. As a result, the presenting function 132 causes the display of the terminal 10 to display the screen expressing the effect of the three-year survival rate as illustrated in FIG. 13A as the relative indexes of the candidate treatments “surgery”, “TAVI”, and “medication”. In this case, for example, when the patient P who thinks the effect in the short term is valuable sees the screen illustrated in FIG. 13A, the doctor and the patient P may agree with each other.

On the other hand, for example, the patient P's doctor may want to show the patient P the screen with the priority on the effect in the long term by changing the outcome. In this case, for example, the patient P's doctor designates “five-year prediction” as the type of the index, and designates two groups of the patient group “with treatment” having been subjected to the treatment such as “surgery”, “TAVI”, or “medication” for the disease and the patient group “without treatment” not having been subjected to the treatment for the disease, as the reference objects using the terminal 10. As a result, the presenting function 132 causes the display of the terminal 10 to display the screen expressing the effect of the five-year survival rate as illustrated in FIG. 13B as the relative indexes of the candidate treatments “surgery”, “TAVI”, and “medication”. In this case, for example, when the patient P who thinks the effect in the long term is valuable sees the screen illustrated in FIG. 13B, the doctor and the patient P may agree with each other.

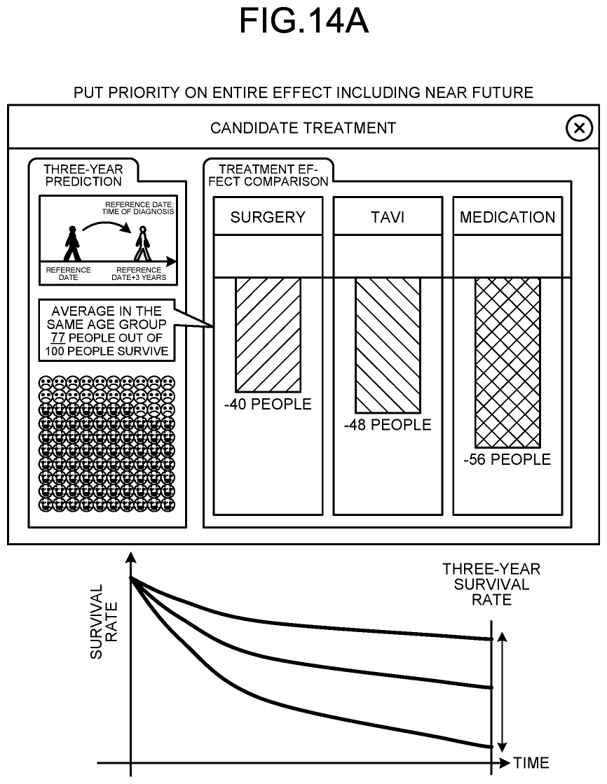

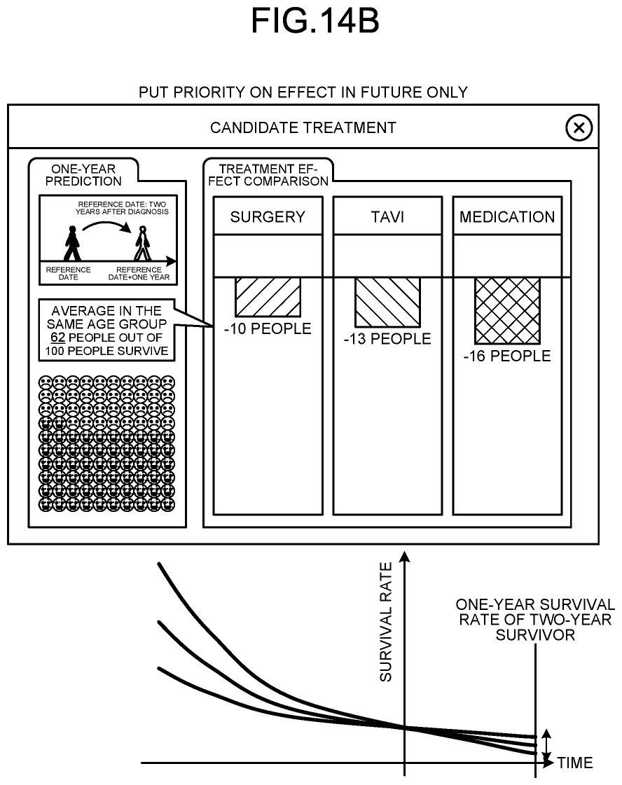

For example, in the case where the patient P's doctor wants to show the patient P the screen with the priority on the overall effect such as “three-year survival rate” as the survival rate, the patient P's doctor designates “three-year prediction” as the type of the index, and designates the patient group “in the same age group” who is in the same age group as the patient P among the patient group “with treatment” having been subjected to the treatment such as “surgery”, “TAVI”, or “medication” for the disease, as the reference object using the terminal 10. As a result, the presenting function 132 causes the display of the terminal 10 to display the screen expressing the effect of “three-year survival rate” as illustrated in FIG. 14A as the relative indexes of the candidate treatments “surgery”, “TAVI”, and “medication”. In this case, for example, when the patient P who thinks the overall effect is valuable sees the screen illustrated in FIG. 14A, the doctor and the patient P may agree with each other.

On the other hand, for example, the patient P's doctor may want to show the patient P the screen with the priority on the effect in the long term such as “one-year survival rate (of two-year survivor)” as the survival rate. In this case, for example, the patient P's doctor designates “one-year prediction” after two years from the diagnosis as the type of the index, and designates the patient group in the same age group as the patient P among the patient group “with treatment” having been subjected to the treatment such as “surgery”, “TAVI”, or “medication” for the disease, as the reference object using the terminal 10. As a result, the presenting function 132 causes the display of the terminal 10 to display the screen expressing the effect of “one-year prediction” after two years from the diagnosis as illustrated in FIG. 14B as the relative indexes of the candidate treatments “surgery”, “TAVI”, and “medication”. In this case, for example, when the patient P who thinks the effect in the long term is valuable sees the screen illustrated in FIG. 14B, the doctor and the patient P may agree with each other.

As described above, in the medical treatment support apparatus 100 according to the second embodiment, the reference candidate objects to become the reference objects are extracted from the candidate treatments chosen by the patient P's doctor and the reference object chosen by the patient P's doctor from among the reference candidate objects is set. Thus, the doctor and the patient P's can have the same sense of values as much as possible, so that the disagreement between the doctor and the patient P can be prevented.

Here, in the medical treatment support apparatus 100 according to the second embodiment, by presenting the table in FIG. 9 to the patient P's doctor, the controlling function 131 sets the reference candidate objects chosen by the patient P's doctor as the reference objects from among the relative indexes of the reference candidate objects in the table in FIG. 9; however, the embodiment is not limited to this example.

Upon the reception of the doctor's request “I want to present all the candidate treatments with the same degree of relative index”, for example, the controlling function 131 may choose the reference candidate object that satisfies the request from among the relative indexes of the reference candidate objects in the table in FIG. 9 and set the chosen reference candidate object as the reference object.

Specifically, for example, if the patient P's doctor thinks the surgery of the patient P is the top priority, the doctor designates the information expressing this decision by the terminal 10 and the controlling function 131 chooses the reference candidate object and the type of the index according to the information designated by the terminal 10 from among the relative indexes of the reference candidate objects in the table in FIG. 9. For example, the controlling function 131 chooses “one-year prediction” as the type of the index and two groups of the patient group “with treatment” having been subjected to the treatment such as “surgery”, “TAVI”, or “medication” for the disease and the patient group “without treatment” not having been subjected to the treatment for the disease, as the reference objects. In this case, the controlling function 131 sets the chosen reference candidate objects as the reference objects, and derives the values of the indexes of the reference candidate objects as the relative indexes. As a result, by causing the terminal 10 to display the screen with emphasis on the effectiveness of the surgery as the relative indexes of the candidate treatments “surgery”, “TAVI”, and “medication”, the presenting function 132 presents the screen to the patient P and the patient P's doctor. In this case, when the patient P who is negative about having the surgery sees the screen with emphasis on the effectiveness of the surgery, the doctor and the patient P may agree with each other.

For example, in the case where the patient P's doctor thinks that the medication is effective to the patient P who is not physically very strong, for example, a child or an elderly person, the doctor designates the information expressing this decision by the terminal 10 and the controlling function 131 chooses the reference object and the type of the index according to the information designated by the terminal 10 from among the relative indexes of the reference candidate objects in the table in FIG. 9. For example, the controlling function 131 chooses “one-year prediction” as the type of the index and the patient group “in the same age group” who is in the same age group as the patient P among the patient group “with treatment” having been subjected to the treatment such as “surgery”, “TAVI”, or “medication” for the disease as the reference object. In this case, the controlling function 131 sets the chosen reference candidate objects as the reference objects, and derives the values of the indexes of the reference candidate objects as the relative indexes. As a result, by causing the terminal 10 to display the screen with emphasis on the effectiveness of the medication as the relative indexes of the candidate treatments “surgery”, “TAVI”, and “medication”, the presenting function 132 presents the screen to the patient P and the patient P's doctor. In this case, when the patient P who is not very strong physically but wants to have the surgery sees the screen with emphasis on the effectiveness of the medication, the doctor and the patient P may agree with each other.

Alternatively, the controlling function 131 may choose automatically the reference object and the type of the index from the tendency of the doctor corresponding to the user (patient P's doctor) or the team to which the doctor belongs.

Specifically, for example, in the case where the controlling function 131 refers to the past history of the user's choices and finds out that user tends to choose the surgery as the attribute of the patient, the controlling function 131 automatically chooses the reference candidate object and the type of the index from among the relative indexes of the reference candidate objects in the table illustrated in FIG. 9. For example, the controlling function 131 chooses “one-year prediction” as the type of the index and chooses automatically the patient group “with treatment” having been subjected to the treatment such as “surgery”, “TAVI”, or “medication” for the disease as the reference object. As a result, the presenting function 132 causes the terminal 10 to display the screen with emphasis on the effectiveness of the surgery as illustrated in FIG. 11A, for example. In this case, when the patient P sees the screen with emphasis on the effectiveness of the surgery, the doctor and the patient P may agree with each other.

Here, in the case of the aforementioned automatic choice, the controlling function 131 sets the reference object in accordance with the doctor (or the team to which the doctor belongs); however, the reference object may be set in accordance with the patient, or the reference object may be set in accordance with the combination between the doctor and the patient.

Note that the components of the devices in the drawings in the present embodiment are functional, and do not necessarily need to be physically configured exactly as illustrated in the drawings. That is to say, the specific mode of the dispersion or integration of the devices is not limited to the mode illustrated in the drawings, and a part of or all of the devices may be dispersed or integrated functionally or physically in an arbitrary unit in accordance with various loads, use circumstances, and the like. In addition, each processing function performed in each device can be achieved in an arbitrary part or entirely by the CPU and the computer program analyzed and executed in the CPU, or can be achieved as the hardware by wired logic.

The method described in the present embodiment can be achieved by having a computer, such as a personal computer or a work station, execute a prepared computer program. This computer program can be distributed through a network such as the Internet. Note that this computer program may be stored in a computer-readable non-transitory storage medium such as a hard disk, a flexible disk (FD), a compact disc read only memory (CD-ROM), a magneto-optical disk (MO), or a digital versatile disc (DVD) and executed by being read out from the recording medium by the computer.

According to at least one embodiment described above, the disagreement between the doctor and the patient can be prevented.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical treatment support apparatus comprising processing circuitry configured:
to set at least one reference object to be compared with a patient;
to acquire a first index corresponding to an index expressing an influence of each of a plurality of candidate treatments for a disease on the patient, and a second index corresponding to an index expressing an influence of each of the candidate treatments on the reference object;
to derive a relative index of the first index of each of the candidate treatments with respect to the second index; and
to present the relative index of each of the candidate treatments.

2. The medical treatment support apparatus according to claim 1, wherein the processing circuitry sets a type of the index.

3. The medical treatment support apparatus according to claim 1, wherein the processing circuitry derives, as the relative index, any of ratio, dispersion, and difference of a value of the first index of each of the candidate treatments with respect to a value of the second index.

4. The medical treatment support apparatus according to claim 1, wherein the processing circuitry sets a temporal parameter about the index.

5. The medical treatment support apparatus according to claim 1, wherein
upon reception of a change of a type of the index, the processing circuitry acquires the index of the type after the change, and
the processing circuitry derives the relative index about the index of the type after the change.

6. The medical treatment support apparatus according to claim 1, wherein
the processing circuitry derives the relative index about each of reference candidate objects, assuming that a plurality of combinations chosen from a plurality of the reference objects are the reference candidate objects, and
the processing circuitry sets a reference candidate object that suits a purpose of a user as the reference object.

7. The medical treatment support apparatus according to claim 6, wherein
the processing circuitry presents the relative index of each of the reference candidate objects to the user, and
the processing circuitry sets a reference candidate object chosen by the user, as the reference object.

8. The medical treatment support apparatus according to claim 1, wherein the processing circuitry sets the reference object in accordance with any one of a user, the patient, and a combination of the user and the patient.

9. The medical treatment support apparatus according to claim 1, wherein the processing circuitry derives the relative index of the index of each of the candidate treatments after excluding candidate treatments that are unsuitable for the patient.
